(19) United States
(12) Patent Application Publication
(10) Pub. No.: US 2010/0240089 A1
(43) Pub. Date: Sep. 23, 2010

(54) Lead isotope tracer method to determine bone mineral turnover

(76) Inventors: Michael J. Inskip, Ottawa (CA); William I. Manton, Richardson, TX (US)

Correspondence Address: IRA S. DORMAN, 330 ROBERTS STREET, SUITE 200, EAST HARTFORD, CT, 06108, US

(21) Appl. No.: 12/661,226

(22) Filed: Mar. 12, 2010

Related U.S. Application Data
(60) Provisional application No. 61/210,294, filed on Mar. 17, 2009.

(51) Int. Cl. C12Q 1/02; G01N 23/00

(52) U.S. Cl. 435/29; 436/57

(57) ABSTRACT

A method of determining bone mineral turnover in bone of a subject involves determining isotopic ratio of one lead isotope (preferably ²⁰⁶Pb) to other lead isotopes in a biological material (e.g. blood or urine) of a subject taking dietary calcium, comparing the isotopic ratio determined to a control isotopic ratio to determine a change in lead isotopic ratio, and correlating the change in lead isotopic ratio to a change in bone mineral turnover in bone of the subject. The control is a ratio of the one lead isotope to other lead isotopes in the biological material of the subject before the subject took the dietary calcium. The method is especially useful for companies developing products or by regulatory bodies for evaluating health or other types of claims made by makers of various products or neutraceuticals, relating to calcium status or preventing/stemming loss of calcium from bone.

### CROSS-REFERENCE TO RELATED APPLICATIONS

This application claims the benefit of U.S. Provisional Patent Application Ser. No. 61/210,294 filed Mar. 17, 2009, the entire contents of which his herein incorporated by reference.

### FIELD OF THE INVENTION

The present invention relates to methods of determining bone mineral turnover in bone. In particular, the present method relates to the use of lead (Pb) isotopic measurements in biological materials for determining bone mineral turnover.

### BACKGROUND OF THE INVENTION

A specific test, using biological materials such as blood or urine samples, reflecting the resorption or formation of calcium (or other elemental component) in bone mineral is needed for understanding impacts on bone mineral (calcium) as a consequence of diseases or altered physiological states, and to measure response to nutritional interventions, to dietary supplements drugs and neutraceuticals (or lack thereof), and in order to examine mechanisms of effects.

Current reported measurements (in blood or urine) used for biological markers of bone turnover (bone resorption or bone formation) are widespread (Garnero 2008; Eastell 2008; Kleerekoper 2001). Such methods include, for example, staggered X-ray measurements (e.g. Dual-Energy X-ray Analysis (DXA)) of bone mineral density, bone-specific alkaline phosphatase for determining bone formation, and deoxypyridinoline for determining bone resportion. However, such methods are not specific for the mineralized (elemental or calcium) component of bone, so an assumption is presently made that these ‘biomarkers’ also reflect what is occurring in bone mineral (calcium apposition and resorption). Also, a new experimental method just being developed (Rennie 2006) uses a radioisotope of calcium (⁴¹Ca) to label the bone, and then follows release of this isotope over the rest of a person's life. But this technique needs very sophisticated and expensive equipment and, since the Ca isotope is radioactive, it may require special approval for use. It is also unable to ‘retroactively’ label older bone sites which may be particularly important areas, and those about which information on changes in bone mineral of the skeleton may be required.

Confidence in the results from the use of current markers of bone turnover has limitations (especially in the absence of serial X-ray bone density information) because the data obtained reflect the processes (building up or breaking down) which are part of the organic (non-mineralized or collagen) component of bone. Although a new method exists (described above) for radio-isotopic calcium, which might address the bone mineral component, this new method requires very expensive equipment and uses a radiation emitting isotope for which the ‘degree of comfort’ of patients and physicians may be questionable. Also, no existing method for bone turnover markers can discriminate between turnover changes in a specific skeletal envelope or “compartment” (such as in cortical or trabecular bone) and reflects whole body net changes.

Thus there remains a need for a reliable and simple method of determining calcium turnover in bone.

### SUMMARY OF THE INVENTION

It has now been found that changes in lead isotopic ratios in biological materials of a subject taking dietary calcium provides an accurate and reliable measure of bone mineral turnover in bone of the subject.

Thus, in one aspect of the invention, there is provided a method of determining bone mineral turnover in bone of a subject comprising: determining isotopic ratio of a first lead isotope to other lead isotopes in a biological material of a subject taking dietary calcium; comparing the isotopic ratio determined in (a) to a control isotopic ratio to determine a change in lead isotopic ratio, the control being a ratio of the first lead isotope to other lead isotopes in the biological material of the subject before the subject took the dietary calcium; and, correlating the change in lead isotopic ratio to a change in bone mineral turnover in bone of the subject.

The present method involves measurement of isotopes of lead (Pb) (preferably stable (non-radioactive), naturally-occurring isotopes) present in very small amounts in biological materials (preferably blood or urine) following previous serial ingestion (intentionally or as a result of historic dietary habits) of dietary calcium (for example a calcium-containing food, a calcium-containing beverage and/or a calcium supplement in which minute naturally occurring traces of lead or purposely enriched isotopes of lead are also present. Since many food products (natural and commercially produced) and some neutraceuticals contain calcium, they may also contain minute amounts of Pb isotopes, which may be used as a means of following what is happening to calcium that has been accreted in bone and later released to the circulation.

Most sources of calcium mined for eventual dietary or nutrient purposes that originate from deposited strata will contain small amounts of other elements including Pb, although at extremely low levels which are of no consequence to health. The Pb present, however, is made up of four naturally-occurring, non-radioactive isotopes. According to where in the world the mineral was originally laid down, the composition of three of the four isotopes of Pb may vary slightly, depending on several factors but mainly on the geological age of the nearest Pb-bearing rocks. The present method is based partly on the fact that limestone is enriched in only one lead isotop (²⁰⁶Pb) because the mineral calcite (calcium carbonate) takes in uranium while excluding thorium. Methods presently exist to measure very accurately the Pb present in such calcium minerals, as well as to very precisely measure the proportion of the different Pb isotopes present.

When calcium has been ingested by humans or animals, some is deposited in the skeleton (and so is the Pb) which, over time, is slowly and continually re-released to the blood circulation from the normal functioning of bone cells (modeling). Sometimes, due to illness or disease, this process is sped up or slowed down or otherwise subject to disruption. Further, Pb mimics calcium as to the way it is laid down in the bone mineral (apposition by osteoblasts), i.e. it is incorporated into the mineral matrix through being ‘mistaken’ by the body for calcium. In the same way, when the bone mineral is resorbed by bone cells (osteoclastic activity) the lead, along with the calcium, is released to the circulation. Pb release happens in proportion to the amount of calcium present (since it has come from exactly the same bone resorption sites). Hence, by measuring Pb isotope ratios in biological materials in comparison to that present in a control prior to administration of calcium supplementation, specific information such as the rate at which calcium becomes mobilized (released) from the skeleton, can be obtained on bone mineral resorption (calcium). Preferably, Pb ratios involving ²⁰⁶Pb are measured. Preferably, the biological material is blood or urine, especially blood.

The subject is preferably mammalian, for example, human or non-human animal. The method is particularly effective when the subject is a non-human animal, particularly in a controlled test setting.

In addition, particularly in the case of experimental animal studies, the same exercise (calcium supplementation containing ultra trace deposits of naturally occurring Pb) may be repeated at a later time, in the same test animal, with a different Pb isotopic fingerprint. Information on bone calcium release to blood can thus be looked at over different time periods, in the same individual and under different test conditions.

In preferred embodiments, the present method involves identification of calcium minerals of a quality which meets (or will meet) requirements for use in dietary supplements or as food additives in mammalian diets. These calcium minerals comprise ultra-trace amounts of either (i) naturally occurring Pb isotopes of a sufficiently different composition to those usually present in calcium-containing foods, or to those already known to be present in the skeleton, or (ii) in the case of experimental studies with animals, a formulated mix of enriched stable Pb isotopes to enhance the differences in isotopic composition from that already present in the existing skeleton. Blood or urine samples are collected for trace Pb analysis, with particular attention to the use of ultra-clean vessels and to contamination containment procedures. Sequential sampling over a period of time and analysis establishes a baseline concentration and isotopic ‘signature’ of the Pb as it becomes mobilized from the skeleton as part of the normal processes of bone cell resorption and accretion of mineral (bone modeling). Administration of the selected calcium mineral (with identified Pb tracer) then commences and sequential sampling of blood to collect samples for analysis is continued. If required, following cessation of administering the mineral, sampling can continue in order to follow a return to a ‘baseline’ (or pre-treatment) state. Analysis at each stage involves the use of thermal ionization mass spectrometry (TIMS) or inductively coupled plasma source mass spectrometry (ICPSMS) to measure Pb concentration and isotope ratios of the four stable isotopes at extremely low levels and at high precision, in accordance with methods generally known in the art (e.g.: Franklin 1997; Inskip 1996).

There is an increasing need for reliable and quantitative information about the flux of calcium between bone and blood, for example with respect to (i) dietary calcium supplements and their effectiveness, (ii) effectiveness of drug strategies and (iii) changes in metabolism associated with skeletal diseases and the aging process. The present method provides a testing technique to address such concerns, and is especially useful by regulatory bodies for evaluating health or other types of claims made by makers of various products or neutraceuticals, relating to calcium status or preventing/stemming loss of calcium from bone. It also offers a simple test based on blood or urine samples for tracking potential problems due to bone mineral loss in people suffering from osteoporosis or from other bone diseases where more is needed to be known about Ca (mineral) loss. The present method addresses such questions using analyses over time of simple body fluids in humans and animals and without the need for use of radio-isotopes.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 depicts a graph showing menses status of cynomolgus monkeys used to validate the method of the present invention.

FIG. 2 depicts a study design for validating the method of the present invention.

FIG. 3 depicts long bone biopsies taken with a trephine to provide cortical and trabecular bone.

FIG. 4A depicts graphs showing the effect of ovariectomy (OVX) on blood lead concentrations in the test monkeys (y-axis scale differences should be noted).

FIG. 4B depicts a scaled-up graph showing the effect of OVX surgery on monkey 592 (peri-menopausal animal B in FIG. 4A).

FIG. 5 depicts a graph showing an example of isotopic Pb signatures for Pb sources in a test monkey.

FIG. 6 depicts a graph showing changes in lead isotope ratio in blood samples collected from −200 days OVX to +300 days OVX for animal 659. Squares are ²⁰⁶Pb/²⁰⁴Pb and circles are ²⁰⁶Pb/²⁰⁷Pb.

FIG. 7 depicts a graph showing measured changes (as a % of baseline) in markers of bone formation (osteocalcin, open circles) and bone resorption (deoxypyridinoline, closed circles) for four of the seven cynomolgus monkeys.

### DESCRIPTION OF PREFERRED EMBODIMENTS

The present method was used to anticipate bone mineral (calcium) changes, as well as blood lead levels, associated with menopause in seven nonhuman primates (pre- and peri-menopausal cynomolgus monkeys). The study also used conventional methods (bone scans and biochemical markers of turnover) to examine changes in the skeleton to validate the efficacy of the present method.

The natural property of metals like lead (Pb) to exist in multiple stable isotope form, allows the creation of unique isotopic tracers to investigate how such elements accumulate in, and are eliminated from, the body. The cohort used were seven older macaques which, during their mid-life, had been administered isotopic Pb over a five-month period, resulting in a skeleton whose bone mineral consisted of a unique lead isotopic ‘signature’. An initial bone biopsy and then frequent sequential blood samples were taken over a period of two years, before and after surgically-induced menopause (ovariectomy (OVX)). Confirmation of bone density changes (‘before and after’) were by dual beam X-ray absorptiometry (DEXA). Total lead and stable Pb isotope measurements were analyzed using thermal ionization mass spectrometry (TIMS) and ‘time-lapse analyses’ of isotope ratio changes was used to interpret the results.

Substantial increases in blood lead (2-3 times) occurred about 60 days following induced menopause, although this was less pronounced in the three peri-menopausal animals. Concurrent changes in stable Pb isotope ratios indicate increased mobilization of bone mineral from ‘older’ skeletal sites, with preliminary confirmation by observed increases in the levels of a marker of bone resorption (deoxypyridinoline). Bone scan determinations by DEXA (six months apart) revealed substantial bone mineral density changes, post-surgery. The complex series of isotope ratio changes (different for each animal, depending on its historic and experimental exposure or “bone lead history”) were interpreted and visualized with a time-lapse presentation approach.

### Methods and Materials:

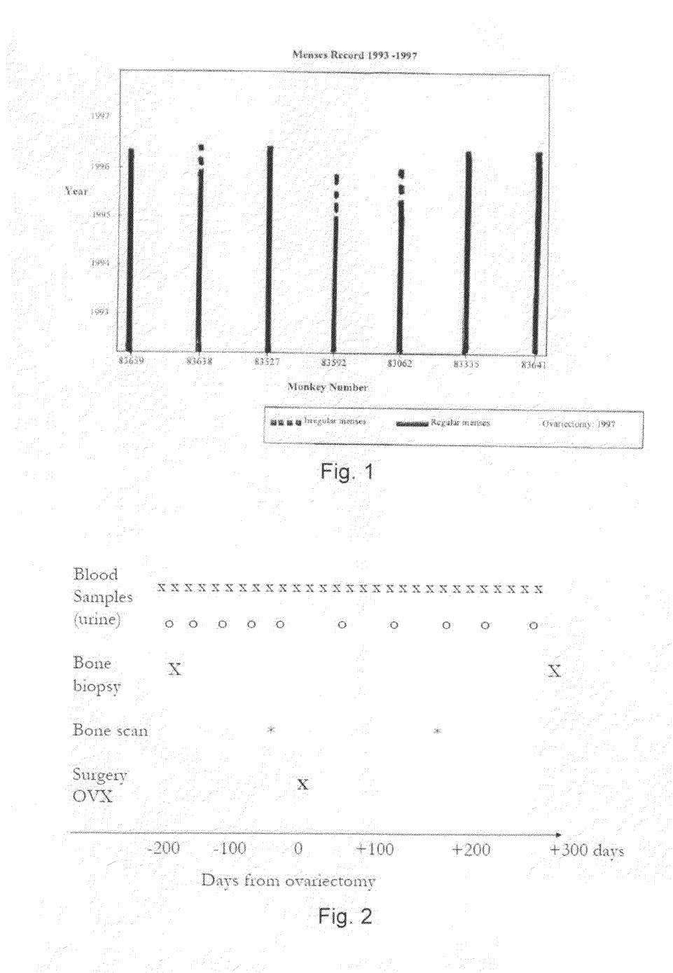

Seven multiparous female cynomolgus monkeys were used in the tests. All animals had an estimated date of birth from 1975 to 1979. They all originated in the Philippines and were shipped to Canada in 1983. From 1983 to 1990 they were all used for breeding. Menses status of the monkeys is shown in FIG. 1. The monkeys were about 20-25 years old. There were four menopausal and three peri-menopausal monkeys. Possible past environmental sources of Pb exposure include diet, air, paint, water, treats, toys, or multiple sources. In 1991, six years prior to this study, the test animals were dosed with common Pb (and some with enriched ²⁰⁴Pb) at 1500 μg/kg bw/day during their final pregnancy from GD 30 to GD 150 (Franklin 1997). Peak blood lead values were 10-50 μg/dL but had long since returned to low levels (or near background) for the current study (0.5 to 5 μg/dL).

The study design is illustrated in FIG. 2. Blood and urine samples were taken under Class-100 clean room conditions into polytetrafluoroethylene (PTFE) containers at intervals over an almost two year period from 200 days pre-ovariectomy to 300 days post-ovariectomy. Lead was extracted from samples chemically (Franklin 1997; Inskip 1996). Pb and Pb isotope analysis was done using a Finnigan MAT 261 thermal ionization mass spectrometry (TIMS) (Thermo Fisher Scientific Inc., Waltham, Mass.), which provides highly precise isotope ratios and accurate total lead concentrations (Manton 2005).

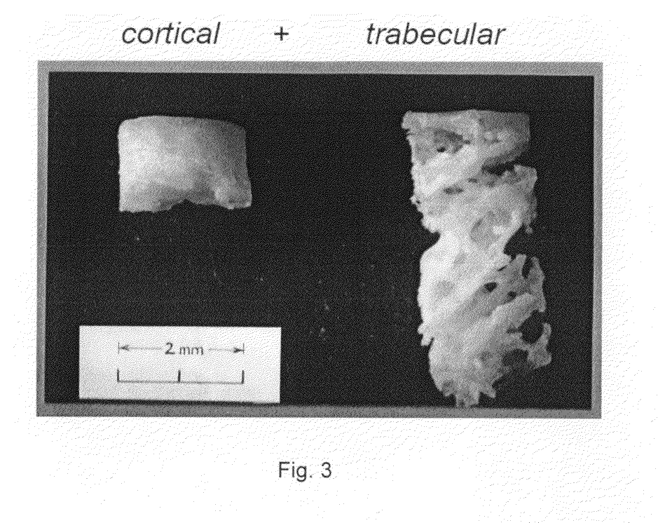

Bone biopsies were taken with a trephine from the ends of long bones almost 200 days pre-ovariectomy and 300 days post-ovariectomy to provide trabecular and cortical bone (see FIG. 3). Bone scans were taken by DXA (Hologic QDR 4500A bone densitometer, Hologic Inc., Bedford Mass.) pre-ovariectomy and post-ovariectomy six months apart. Surgical ovariectomy (OVX) was performed in the middle of the study.

### Effect of OVX Surgery on Blood Lead Levels and Isotope Ratios:

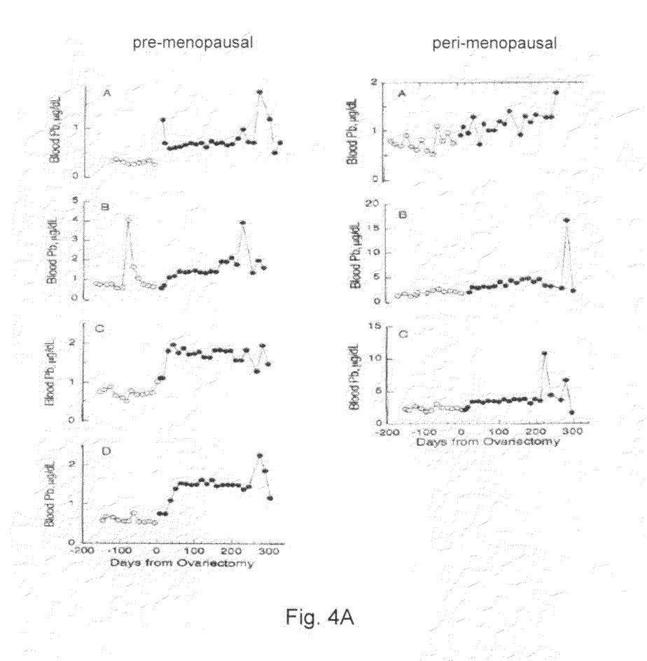

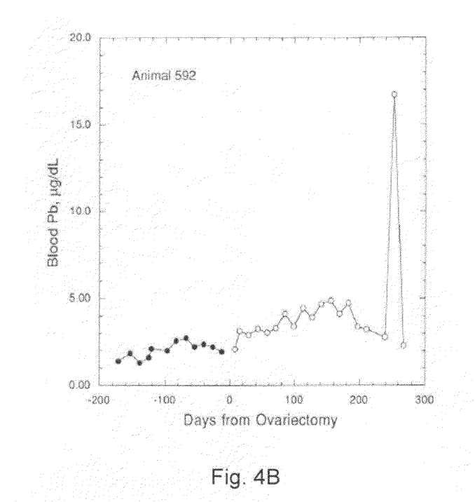

FIG. 4A shows the effect of OVX surgery on blood lead concentrations in the test monkeys. Substantial (2-3 times) increases were seen in blood lead concentrations 60 days following surgery. The increases were less pronounced in those animals previously peri-menopausal. A new “steady-state” blood lead level emerges, except for the period from 250-300 days post-surgery. FIG. 4B is a scaled-up graph showing the effect of OVX surgery on monkey 592 (peri-menopausal animal B in FIG. 4A). FIG. 4B shows a spike in blood lead level at Day 250 post-surgery from about 3 μg/dL to about 17 μg/dL.

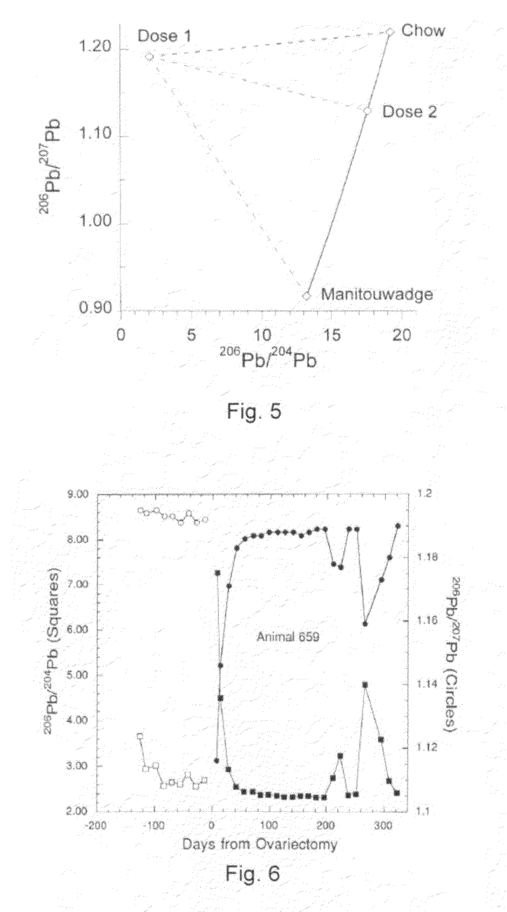

Test monkeys were previously exposed to Pb from a variety of sources. Common Pb is found in diet, water, treats, toys, etc. Enriched ²⁰⁴Pb in a formulated dose was administered during an earlier study with the animals. Manitouwadge Pb (one with a very unusual isotopic signature) was found in deep bone and its source was unknown, although it may have originated from paint or another environmental source (toys) ingested by the animals. Based on this information, an example of isotopic Pb signatures for Pb sources in a test monkey is shown in FIG. 5. The dotted lines in FIG. 5 are “mixing lines” between lead sources. Movement along each line represents relative importance of contribution of lead in blood from Dose 1 and the various sources, for example, from Dose 1 and the Manitouwadge signature (originating from bone).

FIG. 6 shows changes in lead isotope ratio in blood samples collected from −200 days OVX to +300 days OVX for animal 659. Whenever blood lead concentrations changed substantially, measurements of the isotopic ratios (206/204 and 206/207) also changed, and indicated that the source of the lead in bone that was mobilized was coming from a different compartment of the skeleton, sometimes from a component that had not been previously identified, and which probably was in deep cortical bone.

### Effect of OVX Surgery on Bones

In order to correlate the effect of OVX surgery on changes in lead isotope signatures to changes in bone minerals, the effect of OVX surgery on the bones of the test animals was examined.

Substantial changes in the skeletal site of resorption/formation were observed in bones between pre-menopause (i.e. pre-OVX surgery), just post-OVX surgery and 200-300 days after surgery. A sudden switch occurs in this animal, post surgery, and a later adjustment to a new steady pattern, with another shift in re-modeling apparent at Day 200-300 post-OVX surgery. This correlates well with substantial changes in lead isotope ratios at the same times. Thus, changes in lead isotope ratios indicate “new” or different regions of bone are being re-modeled, i.e. it is the concentration of Pb in the bone at the location where Basic Multicellular Unit (BMU) is active that determines (i) Pb returned to the circulation and (ii) the isotopic composition of the Pb. It is apparent that changes in lead isotope ratios are correlated to bone re-modeling and thus to changes in bone mineral turnover.

Bone scans of test animals' skeletons pre-OVX surgery and post-OVX surgery using Dual-Energy X-ray Absorptiometry (DXA) analysis showed quite rapid changes (six months) in bone mineral density (BMD), although less change is seen for the animals already pen-menopausal. Sequential measurements of deoxypyridinoline (D-Pyr) indicated that bone resorption occurred more rapidly post-OVX surgery, and was more marked in the four animals which were originally pre-menopausal. This also correlates well with changes in lead isotope ratios. Table 1 below shows the measurements of lumbar spine bone mass for pre- and post-surgery. In six out of seven animals a reduction was observed.

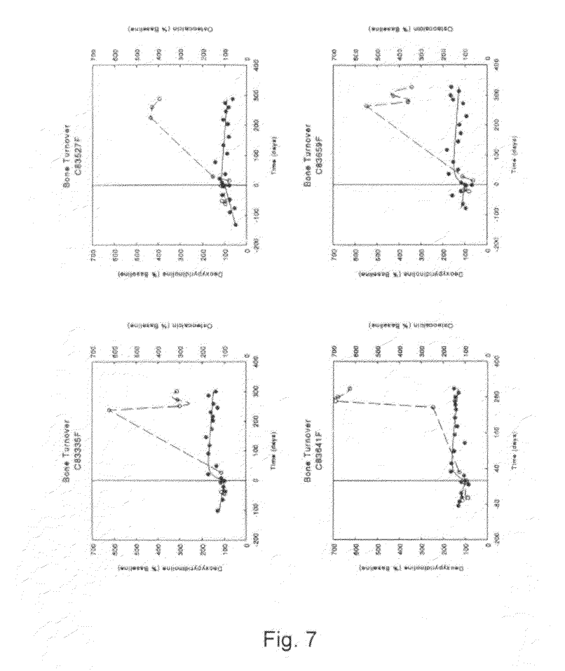

Time sequence analysis of changes in lead isotope ratios using a computer further showed the power of the present method to follow what is happening to bone mineral (presumably Ca) returning to blood in a controlled laboratory setting. As osteoclastic resorption of bone mineral proceeds (3-4 million sites in skeleton) Pb isotopes are transferred to the blood and osteoblastic action in bone formation can then ‘re-incorporate’ Pb from the blood. FIG. 7 shows, for four animals, the bone turnover (% of baseline) pre- and post-OVX surgery, with two markers often used (osteocalcin for bone formation and deoxypyridinoline for bone resorption). Although not easy to interpret due to non-continuous samples, the osteocalcin data suggest spikes of bone formation activity at about the same time as indicated by the bone lead data.

The present method is more representative of bone mineral changes than prior art biomarkers of resorption/formation (e.g. D-Pyr, osteocalcin, BSAP, etc.) since the prior art biomarker methods measure non-mineral parts of the bone.

TABLE 1
Measurements by DXA of lumbar spine bone mass
(and bone mineral Ca) for each animal, before and
six months post-OVX surgery

| | | Lumbar Spine DXA | | |
|---|---|---|---|---|
| | Date pre-/post- | Area | L1-L6 BMD | BMC |
| Animal | OVX surgery | (cm²) | (g/cm²) | (g) |
| C83659F | 08 Apr. 97 | 19.53 | 0.67 | 13.08 |
| | 23 Jan. 98 | 18.49 | 0.61 | 11.20 |
| C83641F | 08 Apr. 97 | 20.76 | 0.71 | 14.68 |
| | 24 Jan. 98 | 19.85 | 0.65 | 12.88 |
| C83592F | 03 May 97 | 21.29 | 0.69 | 14.59 |
| | 23 Jan. 98 | 21.12 | 0.65 | 13.68 |
| C83527F | 03 May 97 | 22.26 | 0.73 | 16.29 |
| | 24 Jan. 98 | 22.10 | 0.65 | 14.40 |
| C83062F | 03 May 97 | 17.52 | 0.59 | 10.41 |
| | 24 Jan. 98 | 17.45 | 0.59 | 10.24 |
| C83335F | 03 May 97 | 18.29 | 0.64 | 11.66 |
| | 24 Jan. 98 | 17.74 | 0.61 | 10.73 |
| C83638F | 03 May 97 | 19.48 | 0.66 | 12.77 |
| | 23 Jan. 98 | 18.94 | 0.59 | 11.20 |

### REFERENCES

The contents of the entirety of each of which are incorporated by this reference.
Eastell R, Hannon R A. (2008) Biomarkers of bone health and osteoporosis risk. Proc. Nutr. Soc. 67:157-162.
Franklin C A, Inskip M J, Baccanale C L, Edwards C M, Manton W I, Edwards E, O'Flaherty E J. (1997) Use of sequentially administered stable lead isotopes to investigate changes in blood lead during pregnancy in a nonhuman primate (Macaca fascicularis). Fundam Appl Toxicol. 39(2):109-119.
Garnero P, Delmas P D. (2008) Investigation of bone: biochemical markers. In: (Eds) M C Hochberg, A J Silman, J S Smolen, M E Weinblatt, M H Weisman. Rheumatology. Ch. 189, pp. 1943-1953, Elsevier, London.
Inskip M J, Franklin C A, Baccanale C L, Manton W I, O'Flaherty E J, Edwards C M H, Blenkinsop J B, Edwards E B. (1996) Measurement of the flux of lead from bone to blood in a nonhuman primate (Macaca fascicularis) by sequential administration of stable lead isotopes. Fund Appl Toxicol. 33(2):235-45.
Kleerekoper M. (2001) Biochemical markers of bone turnover: why theory, research, and clinical practice are still in conflict. Clin Chem. 47: 1347-1349.
Manton W I, Angle C R, Stanek Krogstrand K L, et al. (2005) Origin of lead in the United States diet. Environ. Sci Technol. 39(22):8995-9000.
Rennie G. (2006) Early Detection of Bone Disease. Lawrence Liverpool National Laboratory: Detecting Bone Cancer, S&TR. December, pp 13-15.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A method of determining bone mineral turnover in bone of a subject comprising:
(a) determining isotopic ratio of a first lead isotope to other lead isotopes in a biological material of a subject taking dietary calcium;
(b) comparing the isotopic ratio determined in (a) to a control isotopic ratio to determine a change in lead isotopic ratio, the control being a ratio of the first lead isotope to other lead isotopes in the biological material of the subject before the subject took the dietary calcium; and,
(c) correlating the change in lead isotopic ratio to a change in bone mineral turnover in bone of the subject.

2. The method according to claim 1, wherein the first lead isotope is ²⁰⁶Pb.

3. The method according to claim 1, wherein the first lead isotope and the other lead isotopes are non-radioactive and naturally-occurring.

4. The method according to claim 1, wherein the biological material is blood or urine.

5. The method according to claim 1, wherein the dietary calcium taken by the subject is provided by a calcium-containing food, a calcium-containing beverage, a calcium supplement or a combination thereof.

6. The method according to claim 1, wherein the dietary calcium taken by the subject is provided by a calcium supplement having a different Pb isotopic ratio than in the subject's normal diet.

7. The method according to claim 6, wherein the different Pb isotopic ratio is caused by purposely enriching the dietary calcium with one or more Pb isotopes.

8. The method according to claim 1, wherein determining the isotopic ratio and comparing the isotopic ratio so determined are done a plurality of times over a period of time to provide a change profile of isotopic ratios.

9. The method according to claim 1, further comprising correlating bone mineral turnover to calcium turnover.

10. The method according to claim 1, wherein the subject is a non-human mammal.

11. The method according to claim 1 conducted in a controlled test setting.
